(19) United States
(12) Patent Application Publication
(10) Pub. No.: US 2026/0192030 A1
(43) Pub. Date: Jul. 9, 2026

(54) DISINFECTION/STERILIZATION METHODS THAT ENABLES REUSE OF AN AUTOMATED PERITONEAL DIALYSIS DISPOSABLE

(71) Applicants: Vantive US Healthcare LLC, Deerfield, IL (US); Vantive Health GmbH, Glattpark (Opfikon) (CH)

(72) Inventor: Ramkumar Jeyachandran, Bangalore (IN)

(21) Appl. No.: 19/440,071

(22) Filed: Jan. 5, 2026

(30) Foreign Application Priority Data
Jan. 6, 2025 (IN) 202541001127

(51) Int. Cl. A61M 1/28; A61L 2/07; A61L 2/10; A61L 2/18; A61L 101/36; A61L 103/15; A61M 1/14

(52) U.S. Cl. CPC A61M 1/282; A61L 2/07; A61L 2/10; A61L 2/18; A61M 1/153; A61M 1/1563; A61M 1/1565; A61M 1/159; A61L 2101/36; A61L 2103/15; A61L 2202/11; A61L 2202/122; A61L 2202/14; A61L 2202/15; A61M 2209/10

(57) ABSTRACT

The present disclosure sets forth an automated peritoneal dialysis (“PD”) system, which enables the re-use of certain disposable components. The APD system includes a sterilization source, such as a steamer or a UV radiation light source that sterilizes a disposable filter. During therapy, the disposable filter is connected to a PD fluid line and to a patient's catheter. Post-therapy, the disposable filter is disconnected from the patient's catheter, but remains connected to the PD fluid line, and the disposable filter undergoes steam or UV radiation sterilization.

### CROSS-REFERENCE TO RELATED APPLICATIONS

The present application claims the benefit of Indian Provisional Application No. 202541001127, entitled DISINFECTION/STERILIZATION METHODS THAT ENABLES REUSE OF AN AUTOMATED PERITONEAL DIALYSIS DISPOSABLE and filed Jan. 6, 2025, the contents of which are hereby incorporated by reference in their entirety.

### TECHNICAL FIELD

The present disclosure relates generally to medical fluid treatments, and in particular to dialysis fluid treatments that require the pumping of patient-injectable fluids.

### BACKGROUND

Due to various causes, a person's renal system can fail. Renal failure produces several physiological derangements. For instance, it is no longer possible to balance water and minerals or to excrete daily metabolic load. Additionally, toxic end products of metabolism, such as urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for the replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi-or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days'worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. Automated PD machines, however, perform the cycles automatically, typically while the patient sleeps. The PD machines free patients from having to manually perform the treatment cycles and from having to transport supplies during the day. The PD machines connect fluidly to an implanted catheter, to a source or bag of fresh dialysis fluid and to a fluid drain. The PD machines pump fresh dialysis fluid from a dialysis fluid source, through the catheter and into the patient's peritoneal chamber. The PD machines also allow for the dialysis fluid to dwell within the chamber and for the transfer of waste, toxins, and excess water to take place. The source may include multiple liters of dialysis fluid including several solution bags.

The PD machines pump used or spent dialysate from the patient's peritoneal cavity, through the catheter, to a drain. As with the manual process, several drain, fill, and dwell cycles occur during dialysis. A “last fill” may occur at the end of an APD treatment. The last fill fluid may remain in the peritoneal chamber of the patient until the start of the next treatment, or may be manually emptied at some point during the day.

In any of the above modalities, the automated machine and even manual CAPD operate typically with a disposable set, which is discarded after a single use. Depending on the complexity of the disposable set, the cost of using one set per day may become significant. Also, daily disposables require space for storage, which can become a nuisance for homeowners and businesses. Moreover, daily disposable replacement requires daily setup time and effort by the patient or caregiver at home or at a clinic.

Traditional APD devices with single pass therapy procedures use pneumatic-based non-invasive technology with a cassette, which is disposed of after every use/therapy. Cassette-based systems have limitations in terms of the fluid handling system complexity, manufacturing, and service. For example, sealing the fluid disposable cassette with the pneumatic path via a gasket to provide actuation has proven to be an issue which delays patient therapy start time, and affects the user's experience.

It is desirable to provide an APD machine that reduces disposable waste and that does so in an aseptic manner without needing a cassette.

### SUMMARY

Known automated peritoneal dialysis (“PD”) systems typically include a machine or cycler that accepts and actuates a pumping cassette having a hard part and a soft part that is deformable for performing pumping and valving operations. The hard part is attached to tubes that extend to various bags. The disposable cassette and associated tubes and bags can be cumbersome for a patient at home to load for treatment. The overall amount of disposable items may also lead to multiple setup procedures requiring input from the patient, which can expose room for error.

The APD system and associated methodology of the present disclosure, on the other hand, converts much of the fluid carrying portions of its PD system into reusable components, which are disinfected after treatment. Fluid handling, sensing components and fluid lines within the machine or cycler are reused. Disposable items remaining may include solution bags and associated lines, a patient line, a drain line leading to a drain bag or house drain, a disinfection container or bag and associated line, and possibly an ultrafiltration or sample container. At least some of those disposable components may be disinfected and reused alternatively.

Multi-use or reuse of disposables may aid in reducing therapy time and help to reduce touch contamination from a patient. For example, touch contamination may occur when a user touches sterile parts of the system, such as the filter and/or the connector to a transfer set or catheter, without first disinfecting their hands. Bacteria from the patient's hands may be transferred to the sterile components, which ultimately may encounter the patient's peritoneum. This creates risk of infection, such as peritonitis. When the disposable component remains connected for more than a single use, for example, the patient will only need to connect the catheter line to the already connected disposable component.

A first primary embodiment of the system and method of the present disclosure includes two pumps, for example peristaltic pumps, wherein one pump is dedicated to pumping fresh dialysis fluid to a patient, while the second pump is dedicated to removing used dialysis fluid from the patient. The lines leading to and from the pumps, and the pumping tubes operating with the pumps, are reused and disinfected after treatment. The disposable items that the patient connects for treatment may include first and second dialysis fluid containers or bags, a drain line leading to a house drain or drain bag, and a patient line. In an embodiment, those lines connect in a sterilized fashion to the PD machine or cycler.

The first primary embodiment also includes multiple valves, such as three-way and two-way valves, which may be electrically actuated valves. The valves are positioned to allow fresh dialysis fluid to be drawn from a desired source, to allow dialysis fluid to be pushed to or removed from the patient, and to allow the PD machine or cycler to operate in a treatment mode or a disinfection mode.

The first primary embodiment further includes flow sensors, such as a fresh dialysis fluid flow sensor and a used dialysis fluid flow sensor, which are used to determine solution volumes, such as a volume of fresh fluid delivered to the patient, used fluid removed from the patient and a difference between the two, which is patient ultrafiltration removal. An inline heater may also be positioned adjacent a temperature sensor, which are used for both dialysis fluid heating and disinfection. A pressure sensor and conductivity sensor may be placed near the patient to ensure proper patient filling and draining. The cycler of the first primary embodiment also includes flexible lines or paths, which are used after treatment is completed. As will be illustrated herein, many of the components of the first primary embodiment, such as the heater and temperature sensor, pressure and conductivity sensors, and the flexible lines or paths used after treatment, are also provided with the other primary embodiments discussed herein.

All pumps, valves, heater, and sensors are under control of or output to a control unit, which includes one or more processor and one or more memory. The control unit of the first primary embodiment may be programmed to control the fresh dialysis fluid pump to push fresh dialysis fluid through the heater and past the temperature sensor that verifies proper treatment temperature (e.g., 37° C.). The fresh dialysis fluid is also pumped through the fresh dialysis fluid flow sensor, which indicates a flowrate that is integrated over time to determine and control a total amount of fresh, heated dialysis fluid delivered to the patient for a subsequent dwell. The fresh peristaltic pump provides a smooth delivery to the patient. The downstream pressure sensor may be a backpressure sensor that monitors the patient's intraperitoneal pressure to prevent overfilling and/or overpressurizing the patient. The downstream conductivity sensor may be used to confirm that the prescribed dialysis fluid having the proper mix is being delivered to the patient.

The first primary embodiment also includes an effluent pump, e.g., a peristaltic pump that is smooth and generally continuous, which pulls effluent from the patient. Here, the pressure sensor monitors a negative pressure that the effluent pump applies to the patient to avoid patient discomfort during drain. Used dialysis fluid flows past the conductivity sensor, the output of which can be used to look for certain markers in the effluent or used dialysis fluid (e.g., for peritonitis). The downstream flow sensor indicates effluent flowrate, which is integrated over time to determine a total amount of used dialysis fluid or effluent removed from the patient after dwell. The total amount of effluent removed less the total amount of fresh dialysis fluid delivered to the patient indicates an amount of ultrafiltration (“UF”) removed from the patient over the corresponding dwell period.

During a patient fill phase, the control unit causes one or both of the two fresh dialysis fluid three-way valves to open depending upon which dialysis fluid container is being used. The control unit further causes the patient line valve to be open and the drain line valve to be closed. After the fill phase in which a prescribed amount of fresh dialysis fluid is delivered by the fresh dialysis fluid pump to the patient as measured by the fresh dialysis fluid flow sensor, the control unit proceeds to a dwell phase in which all valves are closed in one embodiment. After the dwell phase, the control unit proceeds to a patient drain phase, wherein the fresh dialysis fluid valves are closed, while the patient line and drain line valves are open. The control unit causes the used dialysis fluid pump to remove a prescribed or minimum amount of used dialysis fluid from the patient (or until the patient is empty) as measured by the used dialysis fluid flow sensor.

After treatment, the system and method of the first primary embodiment of the present disclosure is disinfected. Here, in one embodiment, the dialysis fluid containers are disconnected from the cycler. The patient line is disconnected from the patient and connected instead to the drain line, which may have been disconnected from the drain. Or, one of the patient or drain lines is removed and the other is connected to a connection point of the removed line, completing a disinfection circuit.

The reusable cycler circuit may be heat and/or chemical disinfected. In one embodiment, one of the fresh dialysis fluid valves is connected to a disinfection, e.g., citric acid, container, while the other fresh dialysis fluid valves is connected to a source of purified water. The control unit causes the fresh dialysis fluid pumps to push disinfectant solution from the disinfection source passed the heater, which heats the solution to a sterilizing temperature of, e.g., 90° C. The control unit opens the patient valve to allow hot, e.g., citric acid solution to be recirculated to the drain. The patient valve is then closed, while the fresh dialysis fluid and drain valves are opened to allow the control unit to cause the effluent pump and the fresh dialysis fluid pump to circulate hot disinfectant through the closed loop for a predetermined disinfection time at a predetermined temperature. At the end of disinfection, the drain valve is opened to drain. The control unit switches the valves and operates the pumps so that water from the water source is rinsed through the system to purge disinfectant residuals to drain. The system is then ready for another treatment.

The present disclosure also sets forth an automated peritoneal dialysis (“APD”) system, which includes a disposable component, such as a disposable filter apparatus that may be sterilized and/or disinfected to facilitate reuse of the filter apparatus.

In particular, the present disclosure sets forth a disinfection/sterilization technique including internal steam sterilization after a hot citric disinfection and flush cycle. Specifically, the multi-use APD device is exposed to heat disinfection with citric or other solutions that kills microbes followed by flush cycles with dialysate or water that ensures endotoxins are flushed and biofilm is reduced. Additionally, disposable filters are included as an additional barrier to ensure no microbes or endotoxins enter the patient during therapy. The application of one or more of these techniques allows the disposable component, such as a disposable filter apparatus, to be reused for more than one therapy cycle.

One sterilization and/or disinfection technique of the present disclosure includes internal steam sterilization. A second sterilization and/or disinfection technique of the present disclosure includes external steam sterilization. A third sterilization and/or disinfection technique of the present disclosure includes UV sterilization, and a fourth sterilization and/or disinfection technique includes hot citric disinfection.

In some aspects, which may be combine with any other aspect herein, the techniques described herein relate to a disinfection system of an automated peritoneal dialysis (“APD”) system including: an APD cycler, including: a fluid pump, a source of fresh PD fluid, an inline heater positioned downstream from the source of fresh PD solution and arranged to heat the fresh PD fluid, a fluid line in communication with the source of fresh PD solution, the inline heater, and the fluid pump, and a control unit; a sterilization chamber; a sterilization source housed in the sterilization chamber; and a patient catheter line in communication with a disposable microbial filter and a patient, wherein the disposable microbial filter is detachably connectable to the patient catheter line,, and wherein the disposable microbial filter is configured to be placed in the sterilization chamber during a sterilization process.

In some aspects, which may be combined with any other aspect herein, the techniques described herein relate to a disinfection system, wherein the sterilization source is a steamer.

In some aspects, which may be combined with any other aspect herein, the techniques described herein relate to a disinfection system, wherein the sterilization source is UV radiation.

In some aspects, which may be combined with any other aspect herein, the techniques described herein relate to a disinfection system, wherein the UV radiation is emitted from a single UV light source.

In some aspects, which may be combined with any other aspect herein, the techniques described herein relate to a disinfection system, wherein the UV radiation is emitted from a plurality of LED UV light sources.

In some aspects, which may be combined with any other aspect herein, the techniques described herein relate to a disinfection system, wherein the disposable microbial filter includes a primary filter and a secondary filter in series.

In some aspects, which may be combined with any other aspect herein, the techniques described herein relate to a disinfection system, wherein the primary filter is a microbial filter and the secondary filter is a microbial filter.

In some aspects, which may be combined with any other aspect herein, the techniques described herein relate to a disinfection system, wherein the primary filter is a microbial filter and the secondary filter is an endotoxin filter.

In some aspects, which may be combined with any other aspect herein, the techniques described herein relate to a disinfection system, wherein the disposable filter further includes a bypass cap coupled to the secondary filter.

In some aspects, which may be combined with any other aspect herein, the techniques described herein relate to a disinfection system, wherein the disposable microbial filter includes an inlet valve and an outlet valve, wherein the inlet valve facilitates flow of dialysate towards the patient catheter line and the outlet valve facilitates the flow of used dialysate away from the patient catheter line.

In some aspects, which may be combined with any other aspect herein, the techniques described herein relate to a disinfection system, wherein the disposable microbial filter is housed within the sterilization chamber during sterilization.

In some aspects, which may be combined with any other aspect herein, the techniques described herein relate to a disinfection system, wherein the disposable microbial filter includes a fresh dialysate chamber and a used dialysate chamber that are not in fluid communication.

In some aspects, which may be combined with any other aspect herein, the techniques described herein relate to a disinfection system, wherein the fresh dialysate chamber includes a filter membrane configured to filter microbes from the fresh PD fluid.

In some aspects, which may be combined with any other aspect herein, the techniques described herein relate to a disinfection system of an automated peritoneal dialysis (“APD”) system including: an automated peritoneal dialysis (“APD”) cycler including: a fluid line, a fluid pump configured to pump fluid through the fluid line from a fluid source; a disposable filter in fluid communication with the fluid line, wherein the disposable filter includes: a primary filter including a first chamber and a second chamber, a bypass cap configured to route the fluid from the first chamber to the second chamber to form a circulation loop with the disposable filter and the APD cycler; and a sterilization chamber configured to house the disposable filter.

In some aspects, which may be combined with any other aspect herein, the techniques described herein relate to a disinfection system, wherein the fluid source includes a solution bag containing a disinfectant.

In some aspects, which may be combined with any other aspect herein, the techniques described herein relate to a disinfection system, wherein the sterilization source is a steamer.

In some aspects, which may be combined with any other aspect herein, which may be combine with any other aspect herein, the techniques described herein relate to a disinfection system, wherein the sterilization source is UV radiation.

In some aspects, which may be combined with any other aspect herein, the techniques described herein relate to a disinfection system, wherein the disposable filter is configured to be reused in an APD therapy more than one time.

In some aspects, which may be combined with any other aspect herein, the techniques described herein relate to a disinfection system, wherein the fluid pump is configured to pump the fluid from the fluid source, through the first chamber, the bypass cap, the second chamber, and into the APD cycler.

In some aspects, which may be combined with any other aspect herein, which may be combined with any other aspect, or portion thereof, any of the features, functionality and alternatives described in connection with any one or more of FIGS. 1 to 11 may be combined with any of the features, functionality and alternatives described in connection with any other of FIGS. 1 to 11.

In light of the above aspects and present disclosure set forth herein, it is an advantage of the present disclosure to provide a system and method for disinfection and/or sterilization to facilitate multi-use or reuse of the disposable segment of the line.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1 is a flow schematic view of a first automated peritoneal dialysis (“APD”) cycler embodiment of the present disclosure having fresh and effluent pumps, and which is configured for disinfection post treatment.

FIG. 2 illustrates the APD system of the present disclosure integrated with a steam sterilization chamber for sterilizing a disposable filter.

FIG. 3 is a cross-sectional view of a disposable filter of the present invention.

FIG. 4 is a cross-sectional view of a primary microbial filter of the disposable filter of the present invention.

FIG. 5 is a cross-sectional view of a secondary microbial filter of the disposable filter of the present invention.

FIG. 6 is a cross-sectional view of a one-way fluid valve of the disposable filter of the present invention.

FIG. 7 illustrates the APD system during the therapy state.

FIG. 8 illustrates the APD system during the internal steam sterilization state.

FIG. 9 illustrates the APD system during the external steam sterilization state.

FIG. 10 ILLUSTRATES THE APD SYSTEM DURING A UV STERILIZATION state.

FIG. 11 is a flow diagram of an example procedure to perform a disinfection procedure using the disinfection system of FIGS. 1 to 10, according to an example embodiment of the present disclosure.

### DETAILED DESCRIPTION

### System Overview

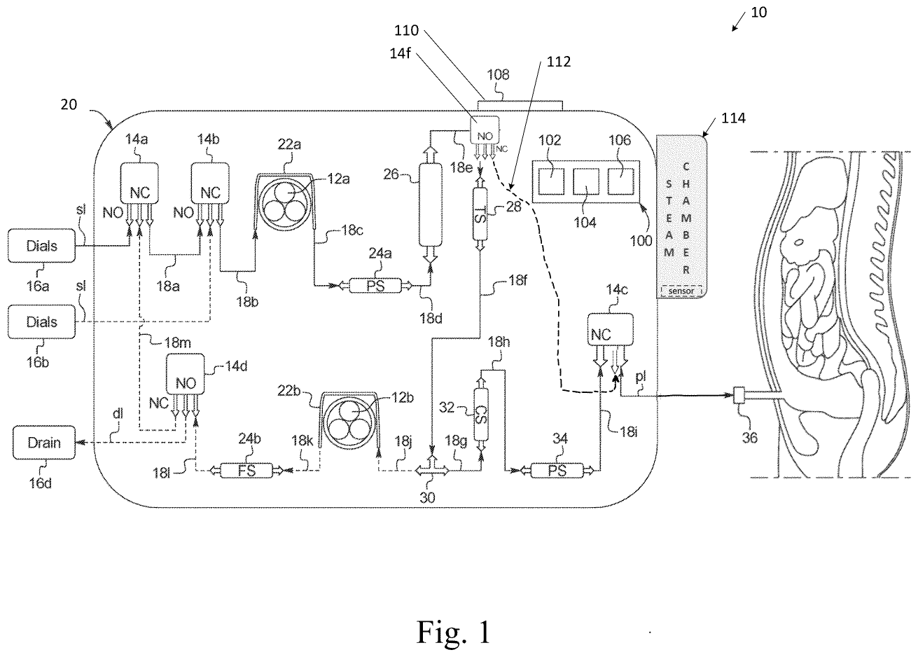

Referring now to the drawings and in particular to FIG. 1, a first primary embodiment of an automated peritoneal dialysis (“APD”) system 10 and associated methodology of the present disclosure includes an APD machine or cycler 20, which is generally defined by the rectangular box in FIG. 1. In the illustrated embodiment, APD machine or cycler 20 includes fresh dialysis fluid pump 12a and used dialysis fluid pump 12b. Pumps 12a and 12b are illustrated as peristaltic pumps, however, pumps 12a and 12b may be any type of fluid pump, for example a gear pump or a membrane pump, and may be of the same or different type. Due to the reusable nature of system 10, pumps 12a and 12b are not limited to types that operate with a disposable item, such as a tube or a flexible chamber. Pumps 12a and 12b instead may include or define internal, e.g., metallic or partially metallic, cavities that receive and contact a fluid to be pumped, such as fresh or used dialysis fluid. On the other hand, pumps 12a and 12b may be peristaltic or membrane pumps that operate with a tube, flexible chamber, or other flexible fluid contacting portion that would in other circumstances be disposable, but which here are disinfected after treatment or prior to a subsequent treatment.

Cycler 20 of system 10 includes a first fresh dialysis fluid valve 14a and a second fresh dialysis fluid valve 14b located upstream of fresh dialysis fluid pump 12a. First fresh dialysis fluid valve 14a is communicated fluidly with a first source of fresh dialysis fluid 16a via a first solution line sl, while fresh dialysis fluid valve 14b is communicated fluidly with a second source of fresh dialysis fluid 16b via a second solution line sl. Dialysis fluid sources 16a and 16b may hold the same or different type of fresh dialysis fluid. Dialysis fluid sources 16a and 16b are premade containers or bags of fresh dialysis fluid in the illustrated embodiment, and solution lines sl may be provided and discarded with sources 16a and 16b. In an alternative embodiment, either one or both sources 16a or 16b is an online peritoneal dialysis fluid source. Additionally, while two sources 16a and 16b and two valves 14a and 14b are illustrated, system 10 alternatively includes only one source and associated valve, or three or more sources and associated valves.

In the illustrated embodiment, valves 14a and 14b are electrically actuated three-way valves, with one port normally open (“NO”), that is, open while no energy is applied, and with another port normally closed (“NC”), that is, closed while no energy is applied. When energy is applied to valves 14a and 14b, the ports switch states such that the NO port closes (restricts flow) and the NC port opens (allows flow). The third port of each valve 14a and 14b is always open and does not switch states. In the illustrated embodiment of FIG. 1, fresh dialysis fluid supply 16a is fluidly communicated via a first supply bag line to NO port of valve 14a, while the third port of valve 14a is fluidly communicated via a reusable line 18a with the NO port of valve 14b. Fresh dialysis fluid supply 16b is fluidly communicated via a second supply bag line to NC port of valve 14b. Thus, when treatment begins, no energy is needed at either valve 14a or 14b to allow flow from first fresh dialysis fluid supply 16a. Here, fresh dialysis fluid pump 12a occludes flow until it is actuated, preventing free flow from fluid supply 16a. After first fresh dialysis fluid supply 16a is consumed and it is time for flow from second fresh dialysis fluid supply 16b, at least valve 14b and perhaps both valves 14a and 14b are energized to close NO port and open NC port of valve 14b to enable pump 12a to pump fresh dialysis fluid from second fresh dialysis fluid supply 16b through valve 14b.

Due to the reuse of system 10, any of the valves described herein, including valves 14a and 14b, may include internal fluid contacting portions that are metallic or otherwise of a nature that would be cost prohibitive to discard after each treatment. In alternative embodiments, any of the valves described herein may operate with tubing (e.g., pinch valves) or flexible membranes (e.g., electric or pneumatic volcano valves), which are disinfected after treatment and reused. In still further alternative embodiments, any of the three-way valves described herein, including valves 14a and 14b, may be replaced via multiple two-way valves.

In FIG. 1, valve 14b is fluidly communicated with reusable fresh dialysis fluid pump line 22a of fresh dialysis fluid pump 12a via reusable line 18b. A reusable line 18c extends from reusable pump line 22a to a fresh dialysis fluid flow sensor 24a, e.g., an inline flow sensor. A reusable line 18d extends from fresh dialysis fluid flow sensor 24 to a dialysis fluid heater 26, e.g., an inline dialysis fluid heater. A reusable line 18e extends from dialysis fluid heater 26 to a dialysis fluid temperature sensor 28, e.g., an inline temperature sensor. A reusable line 18f extends from temperature sensor 28 to one port of a reusable T or Y connector 30. T or Y connector 30 separates a fluid loop (e.g., for disinfection) located to the left of the connector from a series of patient fluid delivery devices to the right of connector 30.

A reusable line 18g leads from connector 30 (to the right) to a conductivity sensor 32, e.g., an inline conductivity sensor. A reusable line 18h leads from conductivity sensor 32 to a pressure sensor 34, e.g., an inline pressure sensor. A reusable line 18i leads from pressure sensor 34 to a two-way normally closed valve 14c, which may be electrically actuated. Valve 14c may have any of the alternative structure and functionality discussed above for valves 14a and 14b. In an embodiment, a patient line pl leads from two-way normally closed valve 14c to a disposable filter 36, which extends to an indwelling catheter located within the patient's peritoneal cavity. Patient line pl may be disposable, e.g., after each treatment using system 10, or reusable as with lines 18a to 18i.

A reusable line 18j leads from connector 30 (to the left) to reusable used dialysis fluid pump line 22b of used dialysis fluid pump 12b. Reusable line 18k leads from used dialysis fluid pump line 22b to a used dialysis fluid flow sensor 24b, e.g., an inline flow sensor. Reusable line 18l leads from used dialysis fluid flow sensor 24b to a three-way drain valve 14d, which may have one port normally open (“NO”), that is, open while no energy is applied, and another port normally closed (“NC”), that is, closed while no energy is applied. Any of the alternative embodiments discussed above for valves 14a and 14b are likewise applicable to drain valve 14d.

In the illustrated embodiment, reusable disinfection line 18m extends from a NC port of drain valve 14d to a NC port of fresh dialysis fluid valve 14a. NO port of drain valve 14d is connected to a drain bag 16d via a drain line dl. Drain line dl may be reusable or disposable. Any of the reusable lines described herein, including reusable lines 18a to 18m, may be metal or plastic, e.g., of a stiffer and more durable plastic than typically used with disposable systems. The plastic is in one embodiment biocompatible, heat-disinfectable and flexible. Pump lines 22a and 22b, patient line pl and drain line dl and disinfection line 18m are likely flexible, e.g., plastic lines. Pump lines 22a and 22b, e.g., peristaltic pump lines, may be different or larger in diameter than the other lines and selected to have a desirable shore hardness for pumping. They may nevertheless be reused according to the disinfection routines discussed herein and likewise be biocompatible, heat-disinfectable and flexible.

Specifications and alternatives for pumps 12a and 12b and valves 14a to 14d are provided above. Flow sensors 24a and 24b as mentioned above may, but do not have to be, inline and invasive flow sensors. Invasive flow sensors or meters 24a and 24b may include rotary vane, vortex shedding, optical, magnetic and mass flow sensors for example. Non-invasive flow sensors may also be provided and include heat pulse, time of flight and optical flow sensors, for example. The outputs from flow sensors or meters 24a and 24b are used as feedback to control pumps 12a and 12b to pump at a desired or specified flowrate, allowing the power delivered to the pumps to be varied as needed. The outputs from flow sensors or meters 24a and 24b are also integrated over time to yield (i) how much fresh dialysis fluid is delivered to the patient, (ii) how much used dialysis fluid is removed from the patient, and (iii) a difference between (ii) versus (i) to know how much ultrafiltration (“UF”) or excess water has been removed from the patient.

Inline heater 26 heats fresh dialysis fluid from its starting temperature to body fluid temperature, e.g., 37° C., for comfortable delivery to the patient. Inline heater 26 may include a flow through and/or circulation heater. The output from temperature sensor 28 located downstream from dialysis fluid heater 26 is used as feedback to control the amount of heating power supplied to heater 26. The feedback allows the target temperature to be reached without significant overshoot. If needed for this or any embodiment discussed herein, an upstream temperature sensor (not illustrated) may be provided, e.g., between flow meter 24a and heater 26, to provide additional feedback, e.g., if incoming fluid to heater 26 is colder than usual then power to the heater is increased. Although not illustrated, an airtrap may be provided to remove air from the fresh dialysis fluid prior to patient delivery. Heating the dialysis fluid tends to separate dissolved air from the dialysis fluid. It is accordingly contemplated to locate the airtrap downstream from heater 26, e.g., along line 18e upstream of temperature sensor 28.

Conductivity sensor 32, e.g., an inline conductivity sensor such as one or more graphite probes, is located between T or Y connector 30 and the patient so as to sense the conductivity of both fresh dialysis fluid (traveling to the patient in a first direction) and used dialysis fluid (traveling from the patient in a second direction). Conductivity sensor 32 is used to sense fresh dialysis fluid to make sure it is the proper type or blend, e.g., if different bagged dialysis fluids are provided for the patient, or if the fresh dialysis fluid is mixed online. Conductivity sensor 32 is used to sense used dialysis fluid to look for solute removal in the patient's effluent (e.g., for urea, β₂ microglobulin, and/or creatinine) or for signs of peritonitis. The flowpath of system 10 is configured such that a single conductivity sensor 32 is able to perform both fresh and used dialysis fluid functions. Conductivity sensor 32 is in one embodiment temperature compensated via the reading from temperature sensor 28. Additional conductivity sensors may be provided in the flowpath of system 10 as desired.

Pressure sensor 34, e.g., an inline pressure sensor, is located in the illustrated embodiment between conductivity sensor 32 and the patient. Pressure sensor 34 may alternatively be a pod pressure sensor or a transducer located within cycler 20, where a pressure transmission tube extends from line 18h/18i to the transducer. The order of conductivity sensor 32 and pressure sensor 34 may be reversed although it may be advantageous to locate conductivity sensor 32 closer to temperature sensor 28. The output of pressure sensor 34 is used to ensure that (i) the positive pressure of fresh dialysis fluid delivered to the patient is within an allowable limit (e.g., 3.0 psig or less) and (ii) the negative pressure of used dialysis fluid removed from the patient is within an allowable limit (e.g., at or between −1.5 psig and zero psig). The flowpath of system 10 is likewise configured such that a single pressure sensor 34 is able to perform both fresh and used dialysis fluid functions. Additional pressure sensors may be provided in the flowpath of system 10 as desired.

Each of pumps 12a and 12b, valves 14a to 14d (and all valves described herein), and heater 26 are powered and controlled via a control unit 100, which includes one or more processor 102, one or more memory 104 and a video controller 106 for controlling a video monitor 108. Video monitor 108 is part of an overall user interface 110 for the system 10 described herein. User interface 110 includes any one or more of a touch screen overlay operable with video monitor 108 and/or one or more electromechanical input device, e.g., membrane switches, for inputting information into control unit. Video monitor 108 and speakers (e.g., operable with a sound card of control unit 100) are provided to output information to the patient or user, e.g., alarms, alerts and/or voice guidance commands.

Similarly, each of flow sensors 24a and 24b, temperature sensor 28, conductivity sensor 32 and pressure sensor 34 outputs to control unit 100. Control unit 100 uses the sensor outputs to control and monitor the components and their functions as described above for system 10 described herein. Control unit 100 is programmed to run any of the flow sequences for the system 10 described herein. Control unit 100 may also include a transceiver and a wired or wireless connection to a network, e.g., the internet, for sending treatment data to and receiving computer readable instructions such as prescription instructions from a doctor's or clinician's server interfacing with a doctor's or clinician's computer.

The system 10 may also include a steam path 112 which, during therapy, aids to regulate temperature and stabilize pressure within the cycler to prevent undesired fluctuations. The system 10 may also include a sterilization or steam chamber 114 that facilitates sterilization procedures as described in more detail below.

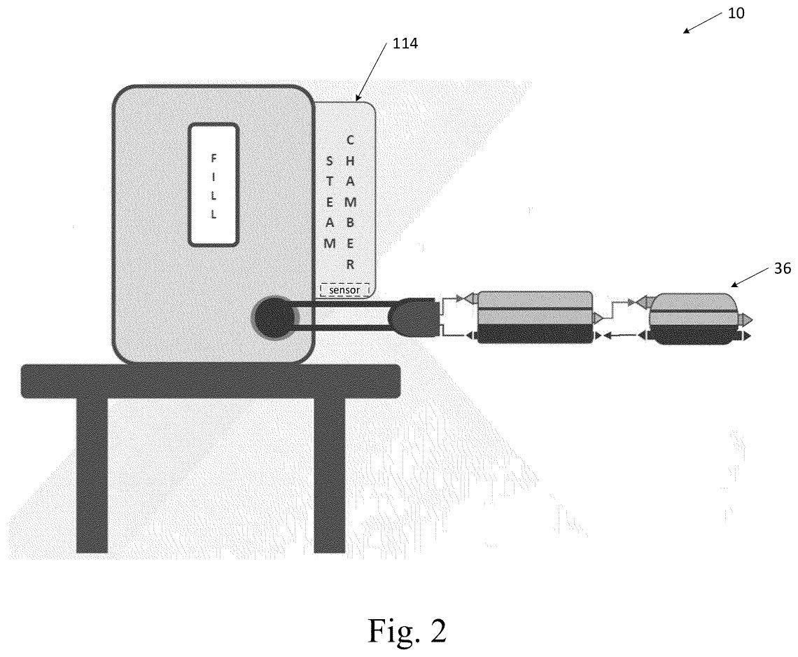

Referring now to FIG. 2, the system 10 may include a disposable filter 36 that may be sterilized via a sterilization chamber, such as the steam chamber 114, integrated in the system 10. The disposable filter 36 is sterilized after the therapy cycle, i.e., post-therapy, and after the citric heat disinfectant, described further below. In particular, the system 10 is exposed to heat disinfectant with citric acid or other solutions that kills microbes followed by flush cycles with dialysate or water that ensures the endotoxins are flushed along with biofilm reduction. At this point, although unlikely, there may be very minimal endotoxins that still exist and, in the therapy phase, these endotoxins may enter the patient leading to complications. Accordingly, the disposable filter 36 is sterilized or disinfected to kill any remaining microbes and to flush out the endotoxins. The disposable filter 36 may be reused more than one time, e.g., the filter may be used for two or more treatments.

### Disposable Filter Design

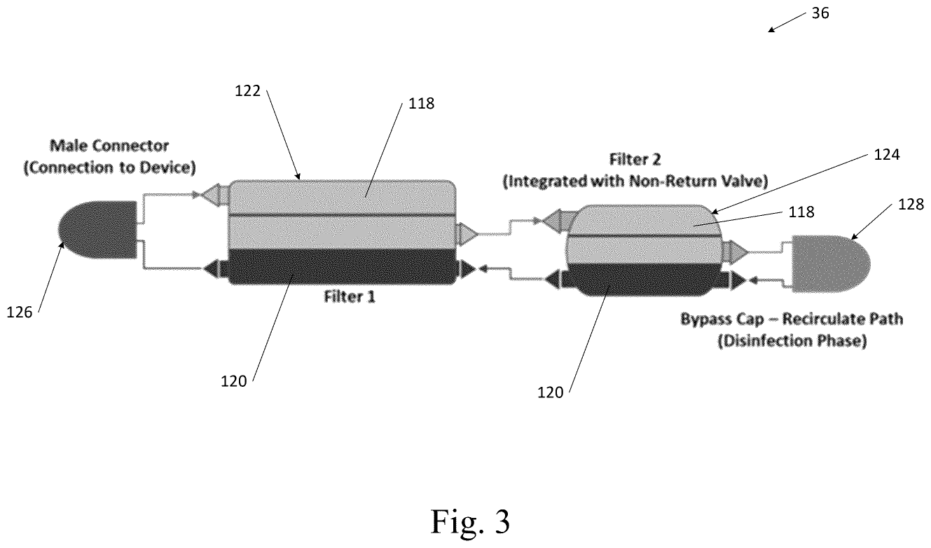

Referring now to FIG. 3, the disposable filter 36 may be connected to a dual lumen or dual tube path (not shown). The dual tube path includes one tube for fresh dialysate and a second tube for used, or spent dialysate. The fresh dialysate flows through the filter membrane 118 in each casing of the disposable filter 36, and the used dialysate exits to a defined fluid path 120 or segment through filters and enters into a used dialysate tube segment. The disposable filter 36 is designed to avoid any contact between the fresh and used dialysate flow path.

Alternatively, the disposable filter 36 and/or patient catheter 140 may be connected to a single lumen tube for fresh dialysate and then connected to single lumen tube for the used, or spent dialysate. For example, during fill, the patient line pl with the disposable filter 36 is connected to the cycler 20 and the patient catheter 140. For drain, the patient line pl is disconnected and a drain line may be connected to the patient's catheter 140. Each of the patient line pl and the drain line dl may include a respective connector for connecting to the patient catheter 140 and/or the disposable filter 36.

As shown in FIG. 3, the disposable filter 36 according to the present disclosure is illustrated. Disposable filter 36 includes a primary microbial filter 122 and a secondary microbial filter 124. The primary microbial filter 122 and the secondary microbial filter 124 each function as an additional barrier to provide a sterile fluid path to avoid the entry of any microbes or endotoxins into the patient. Further, if the primary microbial filter 122 is compromised, or otherwise loses its functionality, the secondary microbial filter 124 continues to function and prevents microbes and endotoxins from entering the patient. In some embodiments, the primary microbial filter 122 and the secondary microbial filter 124 are in series.

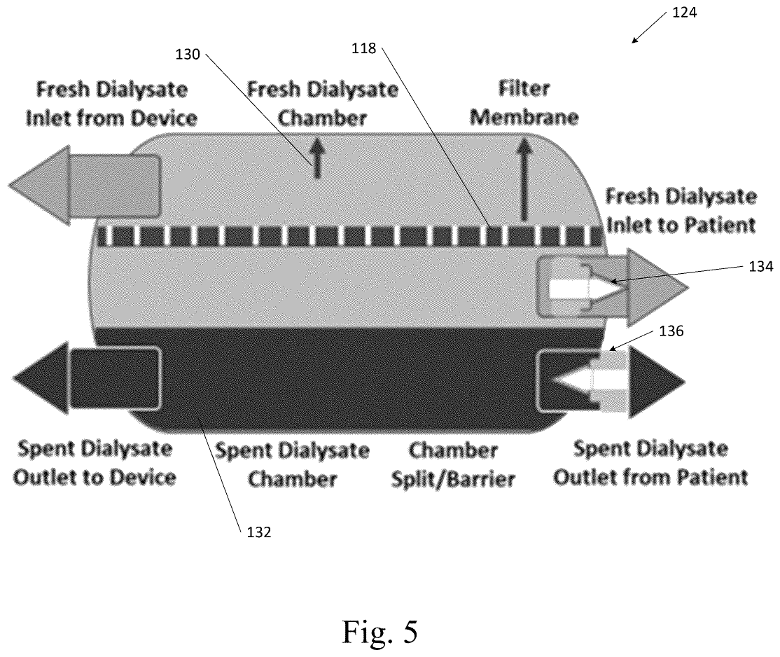

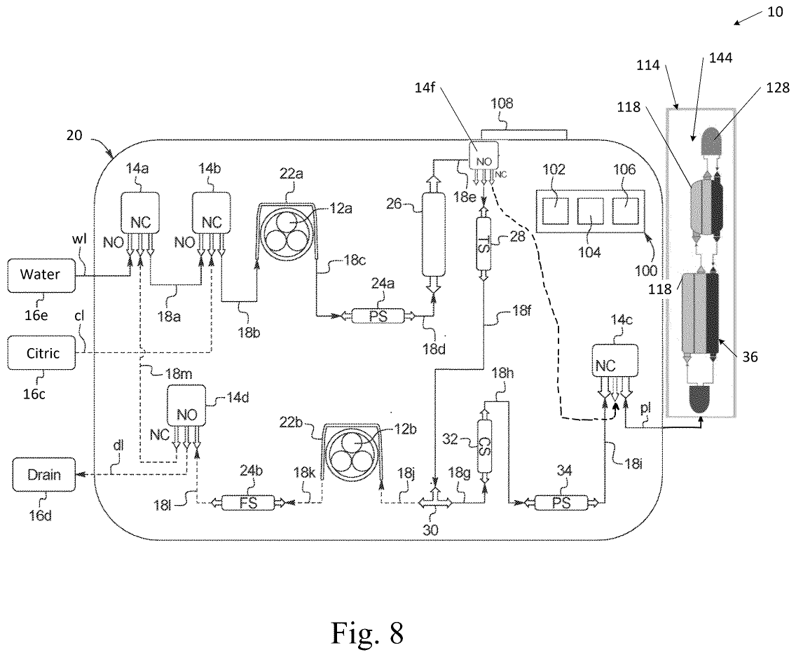

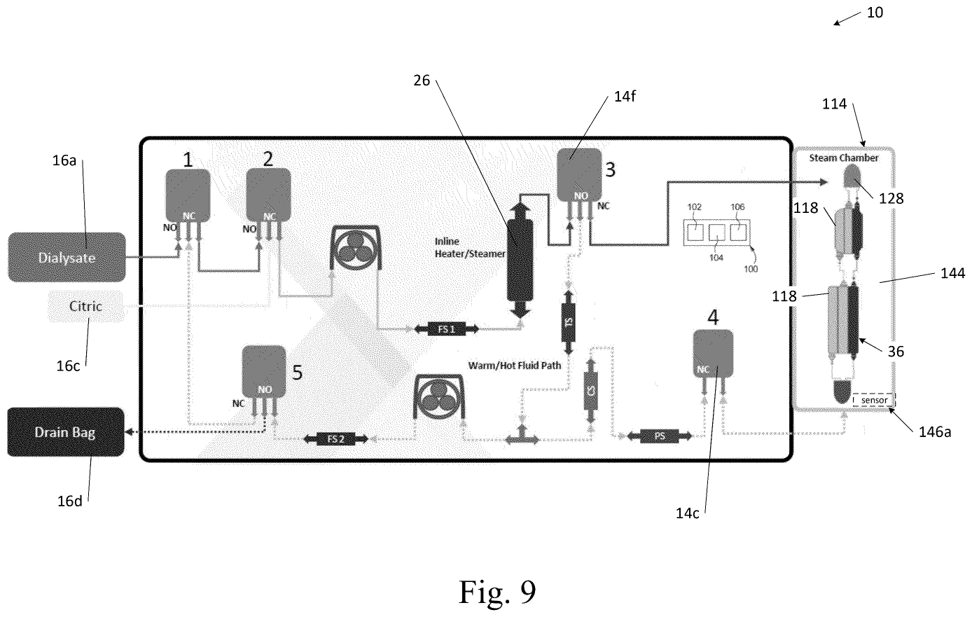

The primary microbial filter 122 of the disposable filter 36 is attached to the system 10 via a male connector 126. The secondary microbial filter 124 is connected to a bypass cap 128 that facilitates the recirculation path during the disinfection phase. The bypass cap 128 in the secondary microbial filter 124 is connected to an inlet valve 134 (as shown in FIG. 5 and to an outlet valve 136 to form a circulation loop (shown in detail in FIGS. 8 and 9). The circulation loop facilitates recirculation of hot disinfection and flush sequences.

Alternatively, the disposable filter 36 may include the primary microbial filter 122 and the secondary filter 124 may be an endotoxin filter that functions as an additional sterile fluid path and functions to prevent microbes and endotoxins from entering the patient during treatment.

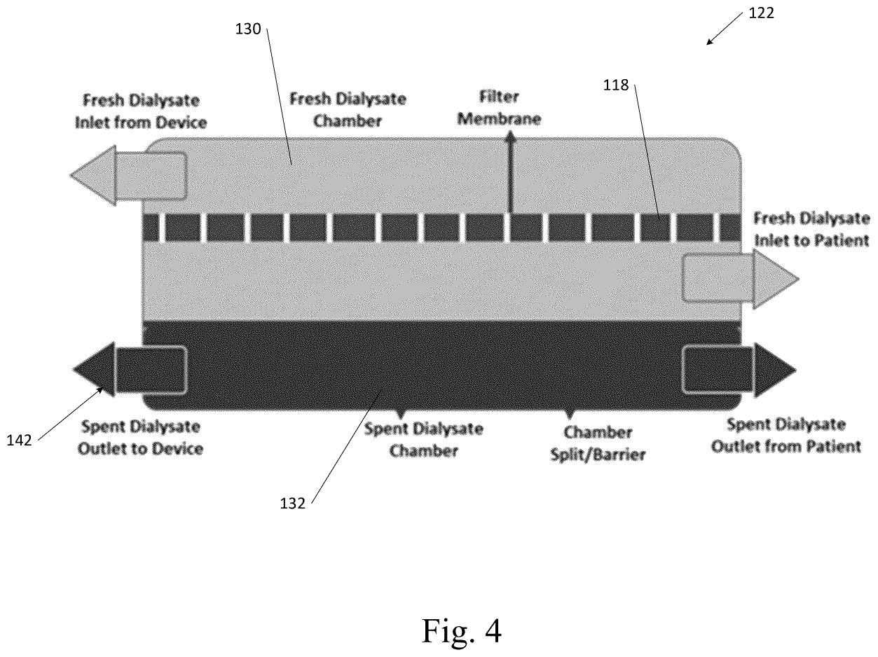

FIG. 4 is a cross-section view of the fluid flow path of the primary microbial filter 122. The primary microbial filter 122 includes a fresh dialysate chamber 130 and/or a used dialysate chamber 132. A barrier separates the two chambers 130, 132, so they are not in fluid communication. Fluid enters the fresh dialysate chamber 130 of the primary microbial filter 122, where it traverses the filter membrane 118 and filters out microbes or endotoxins. The fluid then travels to the patient and/or the secondary microbial filter 124. In some embodiments, effluent from the patient is pumped out of the patient through the used dialysate chamber 132 to the system 10 (not shown) and drained out via the used dialysate outlet valve 142.

FIG. 5 is a cross-section view of the fluid flow path of the secondary microbial filter 124. The secondary microbial filter 124 is located next to the primary microbial filter (shown in FIG. 4) and has a similar construction of the fresh dialysate chamber 130, used dialysate chamber 132, and filter membrane 118 as the primary microbial filter. The secondary microbial filter 124 includes a fresh dialysate inlet valve 134 to the patient, and a used dialysate outlet valve 136 from the patient. The inlet valve 134 and outlet valve 136 may be any suitable shape to facilitate one-way fluid flow, such as a duckbill valve. This secondary microbial filter 124 may alternatively be an endotoxin filter.

Additionally, the inlet valve 134 or outlet valve 136 are configured in such a way that the valves ensure that no used dialysate or effluent enters or touches the fresh dialysate fluid path when the pump from the device sucks the effluent from the patient peritoneum.

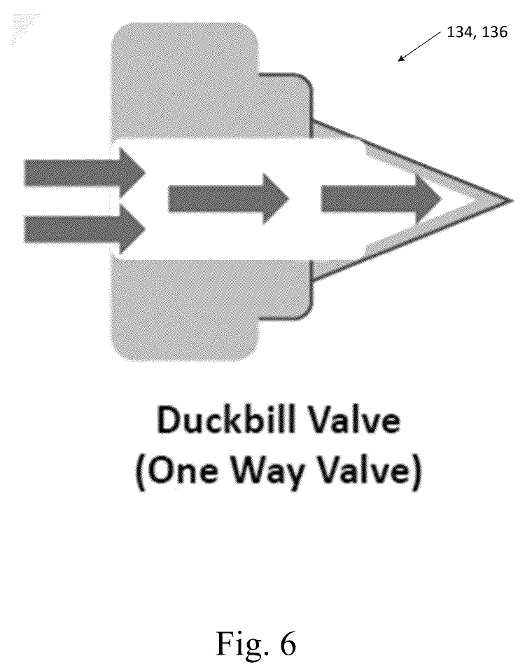

FIG. 6 is a cross-section view of the one-way fluid flow valve 134, 136. For example, the inlet valve 134 may be a duckbill valve that facilitates the flow of fresh dialysate in one direction, i.e., one-way, towards the patient. The outlet valve 136 may be a duckbill valve that facilitates the flow of used dialysate in one direction, i.e., one-way, away from the patient and in the opposite direction of the inlet valve. The valves 134, 136 are, therefore, non-return valves.

### Therapy, Disinfection, and Sterilization Overview

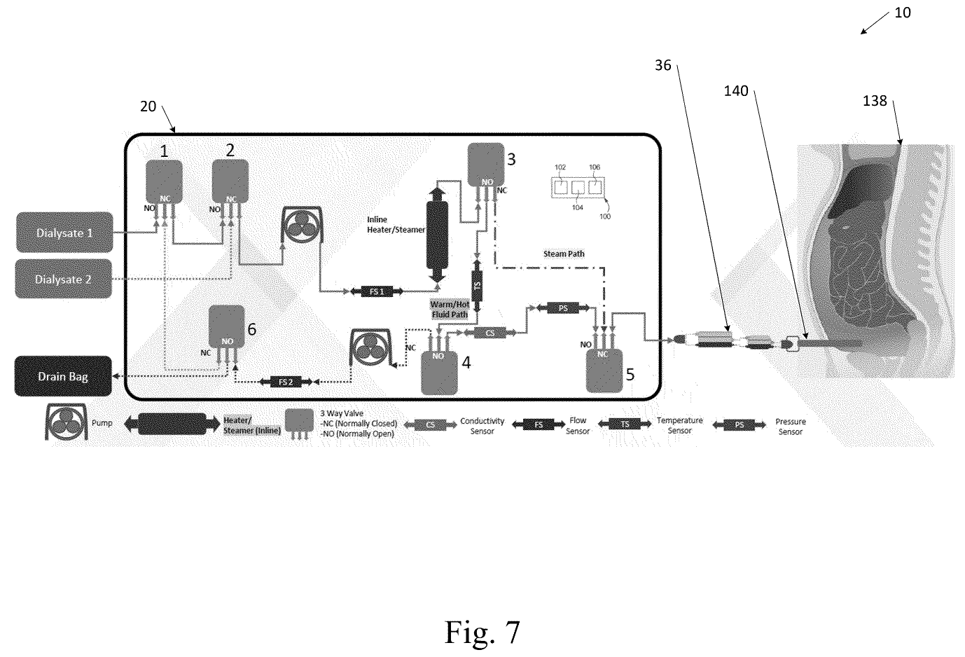

In the therapy state, the disposable filter 36 is connected to the system 10 and to the patient 138 to allow fresh, sterile dialysate to flow into the patient 138 via a patient catheter 140, as shown in FIG. 7. FIG. 7 also illustrates the fluid flow of fresh dialysate from the durable APD cycler 20 through the filter membranes fixed inside the disposable filter housing of the disposable filter 36, which controls and restricts any microbes, endotoxins, or other unwanted particulates from entering the patient 138 during therapy.

FIG. 8 illustrates a chemical disinfectant portion and a purified water rinse portion of a disinfection sequence, respectively, for system 10. Here, second fresh dialysis fluid supply container 16b is replaced with disinfection source 16c, e.g., citric acid, which is placed in fluid communication with the NC port of second fresh dialysis fluid valve 14b via concentrate line cl. First fresh dialysis fluid supply container 16a is replaced with purified water source 16e, which is placed in fluid communication with the NO port of first fresh dialysis fluid valve 14a via water line wl. Drain bag 16d is removed and taken, for example, to a clinic for analysis. Drain line dl is connected instead to a new drain bag 16d. The patient 138 disconnects the disposable filter 36 from the patient catheter and connects it instead to bypass cap 128.

In FIG. 8 with concentrate line cl shown in dotted for citric acid flow and purified water line wl shown solid, control unit 100 actuates fresh dialysis fluid pump 12a to pull disinfectant from source 16c and push same through heater 26, where it is heated to a disinfection temperature of, e.g., 90° C. ±5°C (or as defined for disinfection requirements), through each of the associated valves, lines 18a, 18b, 22a, 18c, 18d, 18e, 18f, 18h, 18i and patient line pl, connector 30 and sensors 24a, 28, 32 and 34 to drain bag 16d. To do so, patient valve 14c is energized open. After a certain amount of heated disinfectant is delivered to drain bag 16d, control unit 100 energizes valve 14a, stopping disinfectant from entering the flow circuit of system 10a, deenergizes (closes) patient valve 14c, and activates both pumps 12a and 12b to recirculate the disinfectant through the entire system for a predetermined amount of time via T or Y connector 30. Heater 26 may continue to heat the disinfectant to maintain the desired disinfection temperature. Three-way valve 14f is maintained to allow disinfection fluid to reach drain valve 14d and drain line dl, and is at some point energized to allow the disinfection fluid to reach and disinfect reusable line 18m. Drain valve 14d is energized to allow disinfection fluid to disinfect drain line dl. Simultaneously, control unit 100 may individually synchronize pumps 12a and 12b on and off as needed to maximize disinfection. Outputs from one or both of flow sensors 24a and 24b may be used to ensure that enough disinfection fluid resides within the closed fluid circuit. If needed, valve 14a may be deenergized (opened) to allow additional disinfection fluid to enter the closed circuit. The above cycles of (i) filling drain bag 16d with heated disinfectant and (ii) recirculating the heated disinfectant throughout the closed circuit may be repeated a predetermined number of times, e.g., three to five times.

In addition to or alternatively, the heat disinfectant process described above with reference to FIG. 8 may use fresh dialysate as the rinse instead of water. The process using of the fresh dialysate rinse is shown in FIG. 9.

As shown in FIGS. 8 and 9, once the therapy sequence is complete, the disposable filter 36 engaged to the patient catheter (shown in FIG. 7) is disconnected, the bypass cap 128 is used to close the connection (as also shown in FIG. 3). The disposable filter 36 remains connected to the system 10 to ensure that the filter 36 undergoes hot disinfection as well. Specifically, while the disposable filter 36 is connected to the system 10, a hot disinfectant, i.e., the citric acid 16c, hot dialysate 16a, or another substantial equivalent, is pumped through the system 10 and flows through the disposable filter 36. The hot disinfectant may be at a temperature of around 80 to 85 degrees Celsius. This disinfection process ensures that the disposable filter 36 is disinfected, and further ensures that any microbes formed inside the system 10 or disposable filter 36 are killed to meet the Log Reduction Value as per standards for patient safety.

After the system 10 and disposable filter 36 are disinfected with the hot disinfectant, the cycler 20 and disposable filter 36 are flushed with water or fresh dialysate to remove and/or drain any remaining microbes, endotoxins, or unwanted particulates inside the system 10. The used disinfectant fluid is drained through the drain bag 16d. After this final rinse, the system 10 and the disposable filter 36 are ready for re-use.

In an alternative embodiment, as shown in FIG. 9, one end of the disposable filter 36 may be connected to a fill line connector and fill line, which is in fluid communication with the hot disinfectant. The other end of the disposable filter may be connected to a drain line connector and drain line, in fluid communication with a drain, such as a drain bag 16d. The hot disinfectant, i.e., the citric acid 16c, hot dialysate 16a, or another substantial equivalent, is pumped through the system 10, through the disposable filter 36, and out through the drain line. This configuration may be used when the cycler 20 includes a single lumen for fill and a single lumen for drain (as opposed to a dual lumen for both fill and drain).

In this configuration, the bypass cap 128 may not be needed, since the disposable filter 36 may not be connected to the system 10 when spent dialysate is being removed from the patient during drain. In some embodiments, the disposable filter 36 may only include the fresh dialysate chamber 130.

Alternatively, or in addition to the hot disinfectant method described above, steam sterilization may be used to provide a more controlled and safe process. FIG. 8 illustrates the process of steam sterilization, which may provide more effective microbe and endotoxin removal than heat disinfectant alone. As further shown in FIG. 8, a heater 26 may be used to warm the solution during the therapy state. Once therapy is completed, the heater 26 heats the disinfectant solution with hot steam produced by the heater 26. Accordingly, after the therapy cycle, after the heat disinfectant cycle, and after the rinse cycle, the steam produced from the inline heater is pushed through valves 14f and 14c, enters the steam chamber 114 and directly contacts exterior surfaces of the filter membrane surface 118 of the disposable filter 36. This inline steam sterilization process ensures that external surfaces of the disposable filter 36, including the bypass cap 28, are sterilized and ready to be re-used for the next therapy cycle.

FIG. 9 illustrates an external steam sterilization process, including a steam bath 144. In particular, after the therapy cycle, after the heat disinfectant cycle, and after the rinse cycle, the steam produced from the inline heater is pushed to the steam bath 144 where the disposable filter 36 has been placed. In some embodiments, other components of the system may be placed in the steam bath 144 for sterilization, including at least a portion of the patient line, a portion of the drain line, and/or tubing or connectors that connect the disposable filter 36 to the patient's catheter and drain line. The hot steam in the steam bath 144 interfaces with the external and internal surfaces of the disposable filter 36 (and/or additional components) that results in disposable sterilization such that the sterilized components (i.e. disposable filter 36) are ready to be re-used for the next day(s) therapy cycle.

The steam chamber 114 may optionally include a sensor 146a for sensing when the disposable filter 36 is placed within the steam chamber 114. The control unit 100 is configured to automatically begin the sterilization process after the sensor 146a is triggered, i.e., when the disposable filter 36 is placed inside the steam chamber 114. The control unit 100 may be time-delayed as to allow the disposable filter 36 to be placed within the steam chamber 114 and to allow the door or opening to the steam chamber 114 to be shut or sealed before the sterilization process begins. The control unit 100 may provide an alarm (e.g., using the user interface 110) indicating when the sterilization process is complete. The alarm may be auditory or visual.

In some embodiments, the control unit 100 prompts a user to disconnect the patient line from the cycler 20 and to insert the patient line into the disinfection chamber 114 prior to sterilization. For example, the control unit 100 can cause the user interface 110 to display a prompt to the user accordingly. The patient line may be externally sterilized or disinfected in the steam chamber 114 as part of the disinfection cycle. The control unit 100 may prompt the user (e.g., using the user interface 110) to insert other parts of the system 10 into the steam chamber for sterilization.

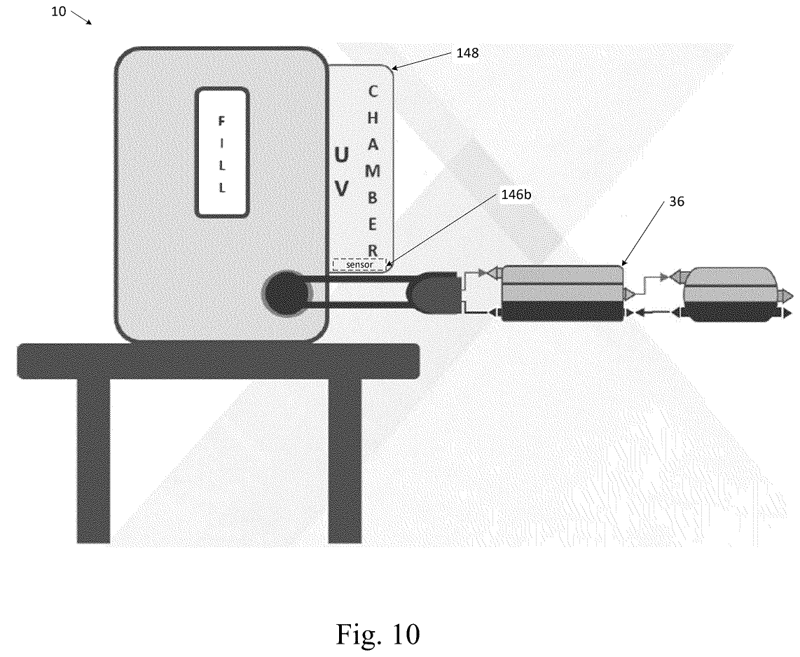

FIG. 10 illustrates UV sterilization of the disposable filter according to an alternative embodiment. The disposable filter 36 may be sterilized using UV radiation from a UV radiation chamber 148. This process occurs after the therapy cycle, after heat disinfectant, and after the rinse cycle. The UV radiation chamber 148 may house a single UV light source or multiple LED light strips that are focused on the multiple surfaces of the disposable filter 36. The heat disinfection and rinse cycle before UV radiation on the disposable filter 36 may effectively clean any soil particulates such that the UV radiation is enhanced because the UV radiation may pass through all surfaces of the filter. The multiple or single source UV can effectively cover all surfaces or walls of the disposable filter 36 to ensure that the disposable filter 36, which is placed inside the UV radiation chamber 148, is effectively sterilized and ready to be re-used for the next therapy cycle. The UV radiation chamber 148 may include a sensor 146b for sensing when the disposable filter 36 is placed within the chamber. The sensor 146b will automatically begin the sterilization process when the sensor 146b is triggered, i.e., when the disposable filter 36 is placed within the UV radiation chamber 148. The sensor 146b may be time-delayed as to allow the disposable filter 36 to be placed within the UV radiation chamber 148 and to allow the door or opening to the UV radiation chamber 148 to be shut or sealed before the sterilization process begins. The sensor may be a temperature, humidity, light-activated, or weight-based sensor. Additionally, the sensor may include an alarm indicating when the sterilization process starts and when the sterilization process complete. The alarm may be auditory or visual.

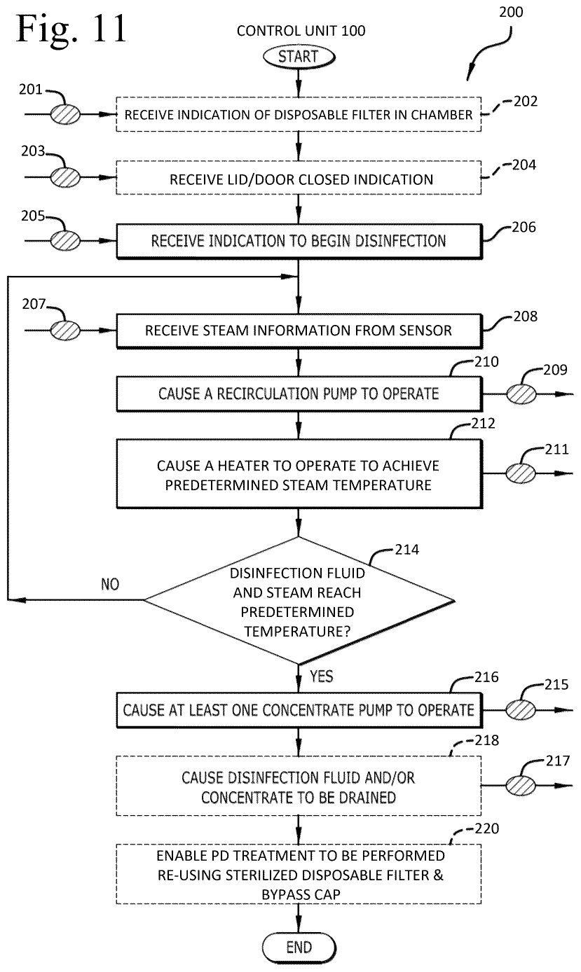

FIG. 11 is a flow diagram of an example procedure 200 to perform a disinfection procedure using system 10 of FIGS. 1 to 10, according to an example embodiment of the present disclosure. Although the procedure 200 is described with reference to the flow diagram illustrated in FIG. 11, it should be appreciated that many other methods of performing the steps associated with the procedure 200 may be used. For example, the order of many of the blocks may be changed, certain blocks may be combined with other blocks, and many of the blocks described may be optional. For example, steps for adding a disinfection fluid to the system 10 may be added. Moreover, the sterilization procedure may be sterilization via UV radiation as opposed to the described steam bath procedure. The operations described in the procedure 200 are specified by one or more of computer readable instructions and may be performed among the control unit 100, and the system 10 more generally.

The example procedure 200 is described for embodiments where a disposable filter undergoes external sterilization via a steam bath. The steps of the procedure 200 are similar when the disposable filter undergoes sterilization via UV radiation. The example procedure 200 begins when the control unit 100 receives a first indication message 201 that the disposable filter 36 is placed inside the sterilization chamber 114 (block 202). The indication message 201 may be received via a user interface that is operable with the control unit 100. For instance, the user interface may include a display screen of the system 10. The user interface may also include a smartphone or tablet computer that is communicatively coupled to the control unit 100 via a direct or network connection.

Returning to FIG. 11, the control unit 100 next receives a second indication message 203 that the user has closed the lid or door to the sterilization chamber 114 to create a fluid-tight seal in the sterilization chamber 114 (block 204). In some embodiments, the control unit 100 may also receive an indication from a sensor 146a located within the sterilization chamber 114 that the disposable filter 36 has been placed in the chamber 114 to create a fluid-tight seal. The control unit 100 next receives a third indication message 205 that specifies disinfection can begin (block 206). In some embodiments, the control unit 100 may cause the sterilization chamber 114 to fill to a certain volume with hot steam from the heater 26. The control unit 100 may further actuate one more valves and/or cause the pump 22a to fill the sterilization chamber with hot steam.

The control unit 100 then receives one or more signals 207 from the sensor 146a that are indicative of a temperature and/or humidity level of the steam inside the sterilization chamber 114 (block 208). The control unit 100 causes the pump 22a to operate by sending one or more signals and/or messages 209, which indicates a programmed flow rate or pump speed. In addition, the control unit 100 transmits one or more messages or signals 211 to the heater 26 to cause the heater 26 to warm the disinfection fluid to a programmed (e.g., predetermined) temperature, which produces steam to a predetermined temperature (block 212). The control unit 100 then performs a feedback loop using the one or more signals 207 from the sensor 146a to ensure the steam bath is warmed to the programmed temperature while the pump 22a is operating. The programmed temperature is a temperature between 80° C. and 105° C. The steam produced from the inline heater 26 is pushed through valves 14f and 14c and enters the steam chamber 114 and directly contacts the surfaces of the disposable filter 36 and the bypass cap 128, to externally sterilize the disposable filter 36.

After the temperature and quantity of the steam has reached the programmed parameters (block 214), the control unit 100 causes the pump 22a to begin pumping concentrate through the disposable fluid line to the durable fluid line by sending one or more control signals 215 (block 216). The control unit 100 maintains this state for a specified time period based on the programmed temperature of the disinfection fluid. The specified time period is at least 10 minutes. During this time, the heated disinfection fluid disinfects outside surfaces of the portion of the disposable filter 36, including the bypass cap 128, contained in the sterilizing chamber 114. In some embodiments, the control unit 100 is configured to send one or more control signals to stop the pump 22a from pumping after the steam has reach the desired quantity. The control unit 100 causes the pump 22a to stop pumping for a defined time period, such as 5 minutes or 10 minutes, to allow the temperature to equalize between the inside and outsides of the disposable filter 36.

After the specified time period has elapsed, the control unit 100 may cause the steam and any fluid concentrate to be pumped to the drain by sending one or more control signals 217 (block 218). Some control signals 217 may cause valve 14c to open while causing the pump 22b to push the disinfection fluid to drain. In some instances, the control unit 100 may cause cold disinfection fluid from the source 16a and/or 16b to push the heated disinfection fluid to the drain and/or cool the disposable filter 36 and the bypass cap 128. Further, after the disposable filter 36 has cooled, some of the control signals 217 may cause the concentrate pump 22a to pump a certain amount of concentrate to push the heated concentrate to drain.

The example control unit 100 next enables a peritoneal dialysis treatment to be performed re-using the sterilized disposable filter 36 and bypass cap 128 (block 220). This may include preparing dialysis fluid in the system 10 by mixing water from the fluid source with one or more concentrates. This may also include warming the mixed dialysis fluid to a patient temperature. This may further include performing one or more fill, dwell, and drain cycles with the prepared dialysis fluid using the system 10.

It should be understood that various changes and modifications to the presently preferred embodiments described herein will be apparent to those skilled in the art. For example, while the fluid schematics illustrated herein show connections to specific NO and NC ports of valves 14a to 14f forming one workable overall flow schematic, the present disclosure is not limited to the specific NO and NC connections, and those of skill may determine others. Also, while a combined chemical and heat disinfection is disclosed, chemical or heat alone may be sufficient. It is therefore intended that such changes and modifications be covered by the appended claims.

The invention is claimed as follows:

1. A disinfection system of an automated peritoneal dialysis (“APD”) system comprising:
an APD cycler comprising:
a fluid pump,
an inline heater positioned downstream from a source of fresh PD solution and arranged to heat the fresh PD fluid,
a fluid line in communication with the source of fresh PD solution, the inline heater, and the fluid pump, and
a control unit;
a sterilization chamber;
a sterilization source housed in the sterilization chamber; and
a patient catheter line in communication with a disposable microbial filter and a patient, wherein the disposable microbial filter is detachably connectable to the patient catheter line, and wherein the disposable microbial filter is configured to be placed in the sterilization chamber during a sterilization process.

2. The disinfection system of claim 1, wherein the sterilization source is a steamer.

3. The disinfection system of claim 1, wherein the sterilization source is UV radiation.

4. The disinfection system of claim 3, wherein the UV radiation is emitted from a single UV light source.

5. The disinfection system of claim 3, wherein the UV radiation is emitted from a plurality of LED UV light sources.

6. The disinfection system of claim 1, wherein the disposable microbial filter comprises a primary filter and a secondary filter in series.

7. The disinfection system of claim 6, wherein the primary filter is a first microbial filter and the secondary filter is a second microbial filter.

8. The disinfection system of claim 6, wherein the primary filter is a microbial filter and the secondary filter is an endotoxin filter.

9. The disinfection system of claim 6, wherein the disposable filter further comprises a bypass cap coupled to the secondary filter.

10. The disinfection system of claim 1, wherein the disposable microbial filter includes an inlet valve and an outlet valve, wherein the inlet valve facilitates flow of dialysate towards the patient catheter line and the outlet valve facilitates the flow of used dialysate away from the patient catheter line.

11. The disinfection system of claim 1, wherein the disposable microbial filter is housed within the sterilization chamber during sterilization.

12. The disinfection system of claim 1, wherein the disposable microbial filter comprises a fresh dialysate chamber and a used dialysate chamber that are not in fluid communication.

13. The disinfection system of claim 12, wherein the fresh dialysate chamber comprises a filter membrane configured to filter microbes from the fresh PD fluid.

14. A disinfection system of an automated peritoneal dialysis (“APD”) system comprising:
an automated peritoneal dialysis (“APD”) cycler comprising:
a fluid line,
a fluid pump configured to pump fluid through the fluid line from a fluid source;
a disposable filter in fluid communication with the fluid line, wherein the disposable filter comprises:
a primary filter comprising a first chamber and a second chamber,
a bypass cap configured to route the fluid from the first chamber to the second chamber to form a circulation loop with the disposable filter and the APD cycler; and
a sterilization chamber configured to house the disposable filter.

15. The disinfection system of claim 14, wherein the fluid source comprises a solution bag containing a disinfectant.

16. The disinfection system of claim 14, wherein the sterilization source is a steamer.

17. The disinfection system of claim 14, wherein the sterilization source is UV radiation.

18. The disinfection system of claim 14, wherein the disposable filter is configured to be reused in a APD therapy more than one time.

19. The disinfection system of claim 14, wherein the fluid pump is configured to pump the fluid from the fluid source, through the first chamber, the bypass cap, the second chamber, and into the APD cycler.

20. The disinfection system of claim 19, wherein the APD cycler further comprises a drain line in fluid communication with the second chamber and a drain, wherein the fluid pump is configured to pump the fluid from the second chamber and into the drain.
